(19) United States
(12) Patent Application Publication
(10) Pub. No.: US 2004/0224399 A1
(43) Pub. Date: Nov. 11, 2004

(54) Recycle soil manufacturing apparatus

(76) Inventor: Matsuro Bundo, Oita-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/609,546

(22) Filed: Jul. 1, 2003

(30) Foreign Application Priority Data
May 8, 2003 (JP) 2003-130046

(51) Int. Cl. C12M 1/00

(52) U.S. Cl. 435/290.4

(57) ABSTRACT

A recycle soil manufacturing apparatus is provided enabling paper/wood chips of finely cut paper and wooden materials to be efficiently changed to soil. A fermenter (1) comprises a paper/wood chip feeder (2) and a fermenter body (10). The paper/wood chip feeder (2) comprises a paper/wood chip supply port (3), a leaf mold supply port (4) and a helical conveyor (5). The fermenter body (10) comprises a helical conveyor (11) transferring paper/wood chips and leaf mold, heaters (14), water sprayers (15), opening/closing type ceiling portions (16) and ventilating fans (17). By being inputted with a signal representing supply rate of the paper/wood chips into the fermenter (1), a control unit controls heating rate of the heaters (14), water spray rate of the water sprayers (15), supply rate of the leaf mold, feed speed of the helical conveyor (11) and opening of the opening/closing type ceiling portions (16) to thereby realize respective predetermined values set corresponding to the supply rate of the paper/wood chips. The paper/wood chips in the fermenter body (10) are quickly changed to soil.

### BACKGROUND OF THE INVENTION

1. Field of the Invention

The present invention relates to a recycle soil manufacturing apparatus by which, where paper or paper and wooden materials (herein collectively referred to as paper/wood materials) are finely cut, shredded or torn to form chips (herein referred to as paper/wood chips), the paper/wood chips are fermented and decomposed by microorganisms and digested by small animals like earth worms, etc. to thereby generate manure or soil (herein collectively referred to as soil).

2. Description of the Prior Art

Nowadays, there are generated a large amount of waste paper/wood materials like waste paper from offices, waste paper or waste corrugated fiberboard of various wrapping or packing, saw dust from saw mills, various wooden materials including pruned or trimmed branches of forest trees, etc. If these waste paper/wood materials are disposed by burning, a large amount of gas like carbon dioxide is generated and this causes problems of global warming, etc. Also, if these waste paper/wood materials are left untouched in the natural state to thereby be returned to soil, it not only takes a long period but also needs a large space of land.

With respect to these waste paper/wood materials like the waste paper from offices, waste paper or waste corrugated fiberboard of various wrapping or packing, saw dust from saw mills, various wooden materials including pruned or trimmed branches of forest trees, etc., although there has been a proposal to finely cut them to be solidified as a solid fuel, this could hardly cope with the advantage of the fossil fuel in the ignitability and combustion operation and there is no substantial demand therefor yet.

Also, there has been a proposal to have the finely cut waste paper/wood materials molded or formed as an integral substance by a binder to be regenerated as construction materials but this is at present still limited to auxiliary uses only.

As the paper and wood are brought up by nutritious matters in the soil, it is preferable to quickly return the waste paper/wood materials to the soil as nutritious matters to thereby prevent the quality of the soil from being deteriorated.

### SUMMARY OF THE INVENTION

It is thus an object of the present invention to provide a recycle soil manufacturing apparatus by which finely cut paper/wood chips are fermented and decomposed by microorganisms and digested by small animals like earth worms, etc. to thereby be recycled to the soil.

In order to solve the abovementioned object, the present invention provides a recycle soil manufacturing apparatus comprising a fermenter having a supply port of finely cut paper/wood chips, a supply port of leaf mold and an opening/closing type ceiling portion, a conveyor conveying in the fermenter the paper/wood chips and leaf mold that have been supplied into the fermenter, a heater heating an interior of the fermenter, a water sprayer spraying water in the fermenter and a ventilation fan ventilating the interior of the fermenter.

According to the recycle soil manufacturing apparatus of the present invention, while the finely cut paper/wood chips are being transferred in the fermenter by the conveyor, they are heated to an appropriate temperature by the heater and are appropriately wetted by the sprayed water from the water sprayer. Also, by the ventilation through the opening/closing type ceiling portion and the forcible ventilation by the ventilation fan, atmosphere in the fermenter is made most suitable for propagation of microorganisms and small animals. Thereby, activities of the microorganisms and small animals are made very active so that fermentation and decomposition by the microorganisms in the supplied leaf mold and digestion by the small animals like earth worms are accelerated and the supplied paper/wood chips can be quickly changed to soil.

It is to be noted that the microorganisms used in the present invention are not only those included in the natural leaf mold but also available from those obtained by cultivation or breeding. In the present invention, the leaf mold is understood to include all these microorganisms.

The recycle soil manufacturing apparatus according to the present invention is preferably constructed to further comprise a control unit inputted with a signal representing a supply rate of the paper/wood chips supplied into the fermenter to thereby control a heating rate of the heater, a water spray rate of the water sprayer, a supply rate of the leaf mold, a feed speed of the conveyor and an opening of the opening/closing type ceiling portion to realize respective predetermined values set corresponding to the supply rate of the paper/wood chips represented by the signal. Then, even if the supply rate of the paper/wood chips supplied into the fermenter changes, the leaf mold supply rate, the conveyor feed speed and the opening of the opening/closing type ceiling portion in the fermenter are correspondingly changed to be automatically set to the respective predetermined optimal values for operation of the fermenter. At the same time, the heating temperature and water spray rate are controlled so that the temperature and humidity in the fermenter becomes the respective predetermined values. Thus, the manufacture of the soil is efficiently carried out.

In this case, a sensor is preferably provided for detecting the temperature and humidity in the fermenter as well as a sensor is preferably provided for detecting the supply rate of the leaf mold, the feed speed of the conveyor and the opening of the opening/closing type ceiling portion and a closed circuit is preferably set up so that the temperature and humidity in the fermenter, the supply rate of the leaf mold, the feed speed of the conveyor and the opening of the opening/closing type ceiling portion, that are detected by the sensors, are controlled to become the abovementioned predetermined values.

Also, the recycle soil manufacturing apparatus according to the present invention is preferably constructed to further comprise a solar generator as an auxiliary electric source. Then, the solar energy is effectively used as a heating source for heat required for fermentation and decomposition of the paper/wood chips. Thereby, the apparatus is economically operated and the paper/wood chips can be efficiently changed to soil.

Moreover, the recycle soil manufacturing apparatus according to the present invention may be so constructed that the soil discharged from the fermenter can be returned to be supplied into the supply port of the leaf mold. Then, if no leaf mold is available, the microorganisms and small animals like earth worms existing in the soil manufactured by the fermenter are effectively used and the paper/wood chips can be quickly changed to soil.

### BRIEF DESCRIPTION OF THE DRAWING

FIGURE 1 is a partly cross sectional longitudinal side view showing a structure of a recycle soil manufacturing apparatus of an embodiment according to the present invention.

### DESCRIPTION OF THE PREFERRED EMBODIMENT

Herebelow, the present invention will be described more concretely based on a recycle soil manufacturing apparatus of an embodiment as illustrated in the appended FIGURE.

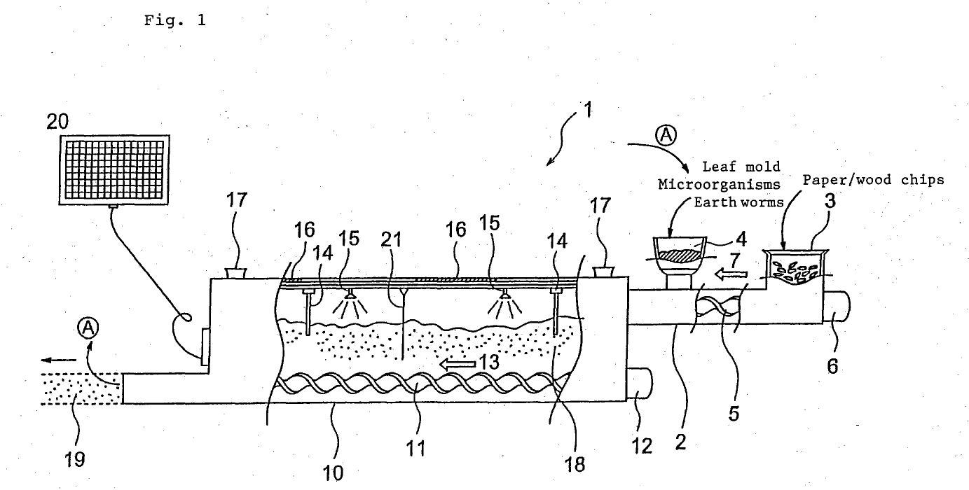

In FIGURE 1, numeral 1 designates a fermenter that comprises a paper/wood chip feeder 2 and a fermenter body 10.

The paper/wood chip feeder 2 comprises a paper/wood chip supply port 3 of finely cut paper/wood chips and a leaf mold supply port 4 provided downstream of the paper/wood chip supply port 3. A helical conveyor 5 is provided in the paper/wood chip feeder 2 to be rotationally driven by a motor 6. By rotation of the helical conveyor 5, the finely cut paper/wood chips and leaf mold that are supplied from the supply ports 3 and 4 are transferred toward the fermenter body 10 in the direction of a white arrow 7 shown by double lines in FIGURE 1.

Within the fermenter body 10, another helical conveyor 11 is provided on an inner bottom portion of the fermenter body 10. When the helical conveyor 11 is rotationally driven by a motor 12, a mixture 18 of the finely cut paper/wood chips and leaf mold that have been fed into the fermenter body 10 by the paper/wood chip feeder 2 is further transferred in the direction of another white arrow 13.

The fermenter body 10 is constructed in the size of length and capacity that enables the finely cut paper/wood chips, while being transferred in the fermenter body 10 by the helical conveyor 11, to be changed to soil by fermentation and decomposition by microorganisms as well as by digestion by small animals such as earth worms or the like.

Within the fermenter body 10, there are provided a predetermined number of heaters 14 for appropriately heating the paper/wood chips and leaf mold in the fermenter body 10 and a predetermined number of water sprayers 15 for appropriately wetting the paper/wood chips and leaf mold in the fermenter body 10. Also, on an upper portion of the fermenter body 10, a predetermined number of opening/closing type ceiling portions 16, made using glass or acrylic resin, are provided, so that, by controlling the opening and closing of the ceiling portions 16, atmosphere in the fermenter body 10 can be maintained in the state most suitable for activities of the microorganisms and small animals like earth worms.

The opening/closing type glass or acrylic resin-made ceiling portions 16 are used such that, if the microorganisms in the fermenter body 10 require sun light for their activities, sun light is taken into the fermenter body 10 through the ceiling portions 16 in the day time and, if the microorganisms in the fermenter body 10 require less sun light, or in the night time, the ceiling portions 16 are closed by sun shades or the like and thereby the sun light is intercepted or the temperature in the fermenter body 10 is adjusted.

Also, in FIGURE 1, numeral 17 designates a ventilation fan that forcibly draws air out of the fermenter body 10 or forcibly takes outside fresh air into the fermenter body 10 so that temperature and humidity in the fermenter body 10 can be adjusted so as to promote the fermentation and decomposition of the paper/wood chips by the microorganisms in the fermenter body 10 or the digestion of the paper/wood chips by small animals like earth worms.

Numeral 19 designates soil that has been manufactured by the fermenter 1. This soil 19 is usable as soil as it is but, for the case where no leaf mold is available to be supplied into the leaf mold supply port 4, the fermenter 1 may be constructed to comprise a conveyor mechanism that conveys a portion of the manufactured soil 19, as shown by reference letters \mathring{A} in FIGURE 1, to be returned for supply into the leaf mold supply port 4. This conveyor mechanism may be appropriately selected from the known art and illustration thereof is omitted for simplicity of description.

Numeral 20 designates a solar generator that functions as an auxiliary electric source for electricity consumed by the fermenter 1, such as a heating electricity of the heaters or the like. Numeral 21 designates a sensor that detects temperature and humidity in the fermenter body 10. Also, while there are provided sensors detecting a supply rate of the leaf mold, a revolution of the helical conveyor 11 and an opening of the opening/closing type ceiling portions 16, these sensors may be appropriately selected from the known art and illustration thereof is omitted.

The operational control of the apparatus is done by a closed circuit control comprising the abovementioned sensors and a control unit (not shown) such that an input signal representing the supply rate of the finely cut paper/wood chips to be supplied into the fermenter body 10 is inputted into the control unit; the control unit makes control so as to realize the supply rate of the leaf mold, the feed speed of the helical conveyors 5, 11 and the opening of the opening/closing type ceiling portions 16 that are previously set corresponding to the input signal of the supply rate of the paper/wood chips; and the control unit also makes control of a heating rate of the heaters 14 and a water spray rate of the water sprayer 15 so that the temperature and humidity detected by the sensor 21 in the fermenter body 10 become the values previously set corresponding to the input signal of the supply rate of the paper/wood chips.

The function of the illustrated embodiment having the construction as described above will be described next. The finely cut paper/wood chips and the leaf mold are supplied into the paper/wood chip supply port 3 and the leaf mold supply port 4, respectively, and, while being mixed together by the helical conveyor 5, they are transferred into the fermenter body 10. In the fermenter body 10, while the paper/wood chips are being exposed to the heat of the heaters 14 and the sprayed water from the water sprayer 15, they are transferred in the direction of the white arrow 13 by the helical conveyor 11.

A signal representing the supply rate of the paper/wood chips into the fermenter body 10 is inputted into a control unit (not shown). The control unit performs a feedback control using detected values by the sensors so that respective predetermined values of the supply rate of the leaf mold, the feed speed of the helical conveyors 5 and 11 and the opening of the opening/closing type ceiling portions 16, that are previously set corresponding to the supply rate of the paper/wood chips, are realized. Also, the control unit, while feeding back the temperature and humidity in the fermenter body 10 detected by the sensor 21, controls the heating rate of the heaters 14 and the water spray rate of the water sprayer 15 so that the temperature and humidity in the fermenter body 10 become the respective predetermined values set corresponding to the supply rate of the paper/wood chips represented by the signal. Thereby, the state in the fermenter body 10 is maintained optimally for activities of the microorganisms and small animals like earth worms that are supplied together with the leaf mold.

If no leaf mold is available, a portion of the soil 19 manufactured in the fermenter body 10 is returned to the leaf mold supply port 4 so that the microorganisms and earth worms, etc. that are needed in the fermenter body 10 are supplied together with the soil 19.

In the day time, heating by the heaters 14 and driving of the motors 6, 12 may be carried out by the electricity generated by the solar generator 20.

In the above, although the invention has been concretely described based on the embodiment as illustrated, the invention is not limited to the embodiment but, as a matter of course, may be added with various modifications within the scope of the invention set forth in the appended claims.

For example, in the recycle soil manufacturing apparatus as illustrated, while the helical conveyors 5, 11 are employed as the conveying devices of the paper/wood chips, etc., not necessarily the helical conveyors but such a known conveying device as a belt type conveyor or a paddle type conveyor may be appropriately selected. Also, in the recycle soil manufacturing apparatus as illustrated, while the bar-like heaters 14 are employed for heating the paper/wood chips, etc. in the fermenter body 10, such a means as feeding warm air into the fermenter body 10, constructing the fermenter body 10 in a jacket type heating wall structure or employing other known types of heaters such as electric heating pipes in the fermenter body 10 can be employed.

Also, while the illustrated recycle soil manufacturing apparatus is described based on the example where the soil is manufactured from the paper/wood chips in which paper and wooden materials are finely cut, if no wooden material is available, the soil may be manufactured from the paper chips only in which waste paper is finely cut or shredded.

1. A recycle soil manufacturing apparatus comprising a fermenter having a supply port of finely cut paper/wood chips, a supply port of leaf mold and an opening/closing type ceiling portion, a conveyor conveying in said fermenter said paper/wood chips and leaf mold that have been supplied into said fermenter, a heater heating an interior of said fermenter, a water sprayer spraying water in said fermenter and a ventilation fan ventilating the interior of said fermenter.

2. A recycle soil manufacturing apparatus as claimed in claim 1, further comprising a control unit inputted with a signal representing a supply rate of said paper/wood chips supplied into said fermenter to thereby control a heating rate of said heater, a water spray rate of said water sprayer, a supply rate of said leaf mold, a feed speed of said conveyor and an opening of said opening/closing type ceiling portion to realize respective predetermined values set corresponding to the supply rate of said paper/wood chips represented by said signal.

3. A recycle soil manufacturing apparatus as claimed in claim 2, further comprising a sensor detecting temperature and humidity in said fermenter, the supply rate of said leaf mold, the feed speed of said conveyor and the opening of said opening/closing type ceiling portion so that the temperature and humidity in said fermenter, the supply rate of said leaf mold, the feed speed of said conveyor and the opening of said opening/closing type ceiling portion are controlled to become said respective predetermined values by a closed circuit.

4. A recycle soil manufacturing apparatus as claimed in claim 1, further comprising a solar generator as an auxiliary electric source.

5. A recycle soil manufacturing apparatus as claimed in claim 1, being so constructed that soil discharged from said fermenter can be returned to be supplied into the supply port of said leaf mold.

6. A recycle soil manufacturing apparatus as claimed in claim 2, further comprising a solar generator as an auxiliary electric source.

7. A recycle soil manufacturing apparatus as claimed in claim 3, further comprising a solar generator as an auxiliary electric source.

8. A recycle soil manufacturing apparatus as claimed in claim 2, being so constructed that soil discharged from said fermenter can be returned to be supplied into the supply port of said leaf mold.

9. A recycle soil manufacturing apparatus as claimed in claim 3, being so constructed that soil discharged from said fermenter can be returned to be supplied into the supply port of said leaf mold.
